(19) United States
(12) Patent Application Publication
(10) Pub. No.: US 2004/0171669 A1
(43) Pub. Date: Sep. 2, 2004

(54) Coated granules based on angiotensin-converting enzyme inhibitor

(76) Inventor: Philippe Chenevier, Montreal (CA)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(21) Appl. No.: 10/477,284

(22) PCT Filed: May 3, 2002
(86) PCT No.: PCT/FR02/01535

(30) Foreign Application Priority Data
May 9, 2001 (FR) 01 06120

(51) Int. Cl. A61K 31/401; A61K 9/26

(52) U.S. Cl. 514/423; 424/469

(57) ABSTRACT

The invention concerns granules based on angiotensin converting enzyme inhibitor, its isomers or its pharmaceutically acceptable salts, characterised in that they are coated and that they contain ACE inhibitor monocrystals, one or several biding agents selected from the group comprising in particular cellulosic polymers, in particular ethylcellulose, hydroxypropylcellulose and hydroxypropylmethyl cellulose, acrylic polymers, polyvidones, polyvinylalcohols, and mixtures thereof, optionally a diluent selected from the group consisting in particular of cellulosic derivatives, starches, lactose, polyols, in particular mannitol, and an antistatic agent selected from the group comprising in particular colloidal silica, precipitated silica and talcum micronized or not. The invention also concerns the method for preparing said granules and orally dispersible tablets wherein they are used.

The present invention relates to coated granules based on angiotensin-converting enzyme inhibitor (CEI) and also to a process for preparing them and to orodispersible tablets comprising said coated granules.

For the purposes of the present invention the phrase “orodispersible tablets” refers to a tablet capable of undergoing disaggregation in the mouth in less than 60 seconds, preferably in less than 40 seconds in contact with the saliva, forming a suspension which is easy to swallow.

Angiotensin-converting enzyme inhibitors (CEI) are used therapeutically in the treatment of essential arterial hypertension and also following myocardial infarction in stabilized patients exhibiting signs of left ventricular dysfunction.

The best-known CEIs are the following: alacepril, benazepril, captopril, ceronapril, cilazapril, delapril, enalapril, enalaprilat, fosinopril, imidapril, lisinopril, moveltopril, perindopril, quinapril, ramipril, spirapril, temocapril, and tandolapril.

These various CEIs are available in the form of tablets or gel capsules.

For example, ramipril is available, depending on country, in the form of a tablet or of a gel capsule, both of which are sold by Aventis under the brand name Altace®.

These forms, owing to their simplicity of use, are ideally suited to ambulatory treatment. However, certain patients, and especially the elderly, experience difficulties of deglutition which are such that it is difficult and, consequently, unpleasant for them to ingest tablets or gel capsules, even together with an intake of liquid.

It is estimated that 50% of the population has difficulties in swallowing tablets or gel capsules. This problem results in the prescribed medicament not being taken and hence in the efficacy of the treatment being severely affected (H. Seager, 1998, J. Pharm. Pharmacol. 50, 375-382).

Although the gel capsule form allows the outer casing (gelatine dome) to be opened and the contents to be administered in a drink or food, the highly unpleasant taste of these CEIs does not hold out a prospect of this mode of administration being satisfactory for patients.

There exists to date no pharmaceutical CEI composition which allows easy administration for patients who have problems of deglutition or for those persons who would like to take their treatment without simultaneous ingestion of liquid.

Rapid-breakdown or fast desintergrating multiparticulate tablets have already been described by the Applicant Company in patents FR 99 02516, FR 97 09233, FR 98 14034, FR 92 08642, and FR 91 09245, but none of these patents describes orodispersible tablets comprising a CEI.

It therefore appeared desirable to remedy this situation and to develop an orodispersible tablet which includes a CEI and whose taste and palatability are entirely acceptable, so that the administration of this tablet is not unpleasant.

The Applicant Company has found that these characteristics were obtained with a tablet based on CEI in the form of coated granules and a mixture of excipients comprising at least one disintegrant, a soluble agent, and a lubricant, and, optionally, a swelling agent, a permeabilizing agent, sweeteners, and flavorings.

In addition such a tablet makes it possible to obtain pharmacokinetic parameters which are equivalent to those obtainable with existing tablets or gel capsules.

The tablets in accordance with the invention exhibit, surprisingly, a pleasant taste, despite the disaggregation of the tablet in the mouth and a release of the active principle which is equivalent to that of the existing forms.

The tablets in accordance with the invention comprise coated granules based on angiotensin-converting enzyme inhibitor, isomers or pharmaceutically acceptable salts of said inhibitor.

The present invention likewise provides these coated granules.

According to one advantageous embodiment of the granules in accordance with the invention the angiotensin-converting enzyme inhibitor is selected from the group consisting in particular of benazepril, captopril, cilazapril, enalapril, sosinopril, lisinopril, perindopril, quinapril, ramipril, and trandolapril, their isomers and their pharmaceutically acceptable salts.

Masking the taste of the CEI is obtained by coating granulated microcrystals of CEI with one or more polymers. The granulometric distribution and mechanical properties of these granules allow their use in the manufacture of rapid-breakdown multiparticulate tablets.

The granules in accordance with the invention based on CEI microcrystals, isomers thereof and pharmaceutically acceptable salts thereof are characterized in that they are coated and in that they comprise

CEI microcrystals,

one or more binders,

optionally a diluent and an antistatic agent.

The majority of CEIs are available in the form of small-sized microcrystals. For example, ramipril is available commercially in the form of microcrystals whose average size is less than 100 μm. The disadvantage of such a granulometry lies in the fact that coating by the methods which consist in spraying these microcrystals with a coating solution or suspension in a fluidized-air bed apparatus is difficult and lengthy.

In order to remedy this drawback, in accordance with the invention, the CEI microcrystals are granulated and coated in such a way as to lead to particles which have a granulometry such that at least 80% of the particles have a size of between 100 and 500 μm and less than 15% of the particles have a size of less than 100 μm.

In the coated granules in accordance with the invention the CEI retains its physicochemical integrity, with granulation and coating not in any way modifying the intrinsic properties of the active principle.

According to the present invention the granules comprise a binder which allows the CEI microcrystals and the other, optional constituents to be bound so as to give granules whose size will facilitate the coating operation.

Said binder may be selected from the group consisting in particular of cellulosic polymers, povidones, polyvinyl alcohols, and acrylic polymers.

Among the cellulosic polymers it will be advantageous to select ethylcellulose, hydroxypropylcellulose (HPC), and hydroxypropylmethylcellulose (HPMC), alone or in admixtures.

Among the acrylic polymers it will be advantageous to select the ammonio-methacrylate copolymer (Eudragit® RL and RS), polyacrylate (Eudragit® NE), and poly-methacrylate (Eudragit® E), Eudragit® being a registered trademark. These polymers are used alone or in admixtures.

The binder is present in proportions which can range up to 15% by weight, preferably up to 10% by weight relative to the weight of the uncoated granules.

Advantageously, in order to promote the granulation of the CEI, a diluent is used. This diluent also makes it possible to increase the proportion of coated granules in the tablets and so to limit the risk of hetero-geneity which is associated with low doses of the CEIs.

Said diluent may be selected from the group consisting in particular of cellulose derivatives and preferably microcrystalline cellulose, starches, lactose, polyols, and, preferably, mannitol.

Said diluent is present in proportions which can range up to 85% by weight, preferably up to 50% by weight relative to the weight of the uncoated granules.

Advantageously, in order to facilitate the fluidization of the material when a fluidized-air bed is used for granulation, it is possible to use an agent which has antistatic properties.

Said antistatic agent may be selected from the group consisting in particular of colloidal silica, particularly that sold under the brand name Aerosil®, and preferably precipitated silica, especially that sold under the name Syloïd® FP244, micronized or non-micronized talc, and mixtures thereof.

Said antistatic agent is present in proportions which can range up to 10% by weight, preferably up to 3% by weight relative to the weight of the uncoated granules.

The granules obtained following granulation are coated by spraying with a coating composition, particularly in the form of a polymer solution or suspension, which will thereby allow the taste of the CEI to be masked.

The coating composition is selected as a function of the physicochemical characteristics of the CEI and is composed of at least one coating polymer and, optionally, an antistatic agent, plasticizers, and soluble agents, especially polyols.

The coating polymer is selected advantageously from the group comprising cellulosic polymers, acrylic polymers, and mixtures thereof.

Among the cellulosic polymers it will be advantageous to select ethylcellulose, hydroxypropylcellulose (HPC), and hydroxypropylmethylcellulose (HPMC), alone or in admixtures.

Among the acrylic polymers it will be advantageous to select the ammonio-methacrylate copolymer (Eudragit® RL and RS), polyacrylate (Eudragit® NE), and poly-methacrylate (Eudragit® E), Eudragit® being a trademark registered by RÖHM.

The coating polymer is present in proportions which can range up to 30% by weight, preferably up to 15% by weight relative to the weight of the coated granules.

The antistatic agent may be selected from the group comprising in particular colloidal silica, especially that sold under the brand name Aerosil®, and preferably precipitated silica, especially that sold under the brand name Syloïd® FP244, micronized or non-micronized talc, and mixtures thereof.

This agent is present in proportions which can range up to 10% by weight, preferably up to 5% by weight relative to the coating polymer.

According to one advantageous embodiment of the invention the polymer constituting the binder of the granule and the coating polymer are the same.

According to one advantageous embodiment the coated granules of the present invention comprise

from 10 to 95% of CEI microcrystals,

from 0 to 85% of a diluent,

from 0 to 10% of an antistatic agent,

from 5 to 50% of a coating polymer,

the percentages being expressed by weight relative to the weight of the coated granule.

Concentrations of diluent of more than 85% are not advantageous from the standpoint of facilitating granulation and result in a substantial dilution of the CEI, and hence the production of a large-sized tablet.

For a coating polymer concentration of less than 5% coating is not sufficient to allow effective masking of the taste. For a concentration greater than 50% the release of the CEI is excessively retarded.

Concentrations of antistat of more than 10% do not bring about any additional improvement in the fluidization of the particles.

According to another advantageous embodiment the coated granules of the invention comprise

from 10 to 95% of CEI microcrystals,

from 0 to 85% of mannitol,

from 0 to 10% of colloidal silica, and

from 5 to 50% of ethylcellulose,

the percentages being expressed by weight relative to the weight of the coated granule.

The invention likewise relates to the process for preparing coated CEI granules.

The process in accordance with the invention comprises in succession the steps

of dry mixing of CEI microcrystals with the diluent and, optionally, an antistatic agent,

of granulation of the mixture obtained in the preceding step by spraying of a solution or suspension of the binder,

of coating of the granules thus obtained by spraying of a suspension of the coating composition,

of drying of the coated granules thus obtained.

According to this process the steps of mixing, granulation, and coating can be carried out in different apparatus or in the same apparatus and in the presence, for each step, of an identical or different mixture of excipients.

In a first embodiment of the granulation process in accordance with the invention the steps of initial dry mixing, granulation, coating, and drying are performed in a fluidized-air bed.

In this case the initial mixture of powder of active principle, diluent, and, optionally, antistatic agent is first of all fluidized before being granulated by spraying onto said powder of a solution or suspension of excipients comprising at least one binder, the grains obtained are subsequently coated by spraying with the suspension of coating composition, the coated granules formed are dried, all of these steps being performed in a fluidized bed.

According to one advantageous embodiment the mixture of excipients employed in the granulation step and the coating suspension employed in the coating step form a single mixture. In this case the step of granulating is distinguished from the step of coating by varying different parameters, such as the spraying rate of the mixture of excipients and the atomizing pressure of said mixture. Accordingly, only part of the mixture of excipients is employed in the granulation step, while the other part is employed in the coating step.

The spraying rate of the suspension of excipients is higher in the granulation step than in the coating step, whereas the atomizing pressure of the suspension of excipients is lower in the granulation step than in the coating step.

In practice, on the laboratory scale in a fluidized-air bed apparatus, for example, of the GLATT GPCG3 type, in the granulation step, the spraying rate of the mixture of excipients is between 15 and 30 grams/minute and the atomizing pressure is between 1 and 2.5 bar.

In the coating step the spraying rate of the coating suspension is between 10 and 25 grams/minute while the atomizing pressure is between 1.5 and 3 bar.

According to one advantageous embodiment from 10% to 30% of the mixture of excipients are pulverized during the granulation step, the remainder to 100% being pulverized during the coating step.

According to this advantageous process, after the active principle, the diluent, and, advantageously, the antistatic agent have been dry-mixed, the fluidized bed is sprayed with a suspension of excipients comprising the coating polymer or polymers and the antistatic agent, the spraying rate and the atomizing pressure of said suspension being varied so as to obtain, first of all, a granulation and, subsequently, a coating of the particles formed.

The coated granules based on CEI that are prepared in this way are intended very particularly to form part of the constitution of rapid-breakdown tablets, which are further provided by the invention.

The tablets according to the invention are intended to undergo disaggregation in the mouth in contact with the saliva in less than 60 seconds, preferably in less than 40 seconds, forming a suspension which is easy to swallow, and are based on coated granules of CEI and a mixture of excipients comprising at least one disintegrant, a diluent soluble agent, a lubricant, and, optionally, a swelling agent, a permeabilizing agent, sweeteners, and flavorings.

The disaggregation time corresponds to the period which elapses between, on the one hand, the moment when the tablet is placed in the mouth in contact with the saliva and, on the other hand, the moment when the suspension resulting from the disaggregation, without chewing, of the tablet in contact with the saliva is swallowed.

According to one advantageous embodiment the tablets in accordance with the invention are based on coated CEI granules, said granules having intrinsic tableting characteristics, and a mixture of excipients, the proportion of mixture of excipients relative to the coated granules being from 0.4 to 10, preferably from 1 to 10, and more preferably still from 1 to 4 parts by weight, the mixture of excipients comprising

a disintegrant,

a diluent soluble agent having binding properties,

a lubricant,

a permeabilizing agent, and

optionally sweeteners, flavorings, and colorants.

The disintegrant is selected from the group consisting in particular of crosslinked sodium carboxymethyl-cellulose, which is referred to in the art by the term croscarmellose, crospovidone, and mixtures thereof.

The diluent soluble agent having binding properties is composed of a polyol of less than 13 carbon atoms which is present either in the form of a directly tabletable product whose average particle diameter is from 100 to 500 μm or in the form of a powder whose average particle diameter is less than 100 μm, said polyol being preferably selected from the group consisting of mannitol, xylitol, sorbitol, and maltitol, with the proviso that the sorbitol cannot be used alone and that, where the diluent soluble agent having binding properties is alone, it is used in the form of a directly tabletable product whereas, where there are at least two diluent soluble agents having binding properties, one is present in the directly tabletable form and the other in the powder form, it being possible for the polyol to be the same, the proportions of directly tabletable polyol and of powder polyol being from 99/1 to 20/80, preferably from 80/20 to 20/80.

The respective proportions of disintegrant and soluble agent employed for the constitution of the excipient are, relative to the mass of the tablet, from 1 to 15%, preferably from 2 to 7% by weight for the former and from 30 to 90%, preferably from 40 to 70% by weight for the latter.

The lubricant is selected from the group consisting in particular of magnesium stearate, stearic acid, sodium stearylfumarate, micronized polyoxyethylene glycol (micronized Macrogol 6000), leucine, sodium benzoate, and mixtures thereof.

The amount of lubricant is from 0.2 to 20 parts per thousand (weight of lubricant/total weight of the tablet), preferably from 5 to 10 parts per thousand.

The lubricant may be distributed within the excipient or, according to one advantageous embodiment, the totality of the lubricant may be distributed on the surface of the tablet.

As permeabilizing agent use is made of a compound selected from the group comprising in particular silicas having a great affinity for aqueous solvents, such as the precipitated silica known better under the brand name Syloïd®, maltodextrins, β-cyclodextrins, and mixtures thereof.

The permeabilizing agent allows the creation of a hydrophilic network which facilitates the penetration of the saliva and hence contributes to better disaggregation of the tablet.

The proportion of permeabilizing agent relative to the mass of the tablet is from 0.5% to 5% by weight.

The sweetener may be selected from the group comprising in particular aspartame, acesulfam potassium, sodium saccharinate, neohesperidine dihydrochalcone, sucralose, monoammonium glycyrrhizinate, and mixtures thereof.

The flavorings and colorants are those commonly used in pharmacy for the preparation of tablets.

The invention will be better understood by means of the remainder of the description which follows, which refers to advantageous exemplary embodiments of the rapid-breakdown CEI tablets in accordance with the invention. It will be appreciated, however, that these examples are given solely by way of illustration, and as advantageous embodiments of the invention, and do not constitute any limitation whatsoever thereon.

### EXAMPLE 1

### Preparation of Coated Granules Based on Ramipril

The granules are advantageously manufactured using a fluidized-air bed granulator, according to the following process.

First of all a polymer solution is prepared by dissolving 109 g of ethylcellulose in 2050 g of isopropyl alcohol.

Subsequently a homogeneous powder mixture consisting of 500 grams of ramipril, 500 grams of mannitol, and 53 grams of silica is fluidized in a fluidized-air bed.

The granulometry of the various powders is less than 100 μm.

Granulation is carried out by spraying the fluidized bed of the homogeneous powder mixture with a first fraction of approximately 15 to 25% of the totality of the polymer solution, for a period of approximately 20 minutes at a spraying rate of approximately 20 to 25 grams/minute under an atomizing pressure of the solution of 2 bar.

This gives a fluidized bed of granules whose granulometry is predominantly between 100 and 500 μm.

The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2.5 bar.

This gives a homogeneous mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 10 mg of ramipril, it will be necessary to take 24.62 mg of the homogeneous mixture of coated granules. The composition of these 24.62 mg is indicated in Table I below:

TABLE I

| | | |
|---|---|---|
| Active principle | Ramipril | 10 mg |
| Diluent | Mannitol | 10 mg |
| Binder/coating | Ethylcellulose | 3.12 mg |
| agent | | |
| Antistatic agent | Precipitated | 1.5 mg |
| | silica | |
| | TOTAL | 24.62 mg |

### EXAMPLE 2

### Preparation of Rapid-breakdown Multiparticulate Tablets Containing 10 mg of Ramipril

Tablets are prepared which contain 10 mg of ramipril and whose composition is evident from Table II below.

Amounts of each of the excipients identified in Table II below, taken in the respective proportions corresponding with the proportions resulting from said table, are screened in bulk on a screen having a mesh aperture of approximately 800 μm.

Subsequently, in a dry mixer, an amount of coated granules corresponding to the proportion of granules which results from Table II is mixed homogeneously with the screened mixture of excipients.

TABLE II
Composition of the tablet containing 10 mg of ramipril

| | | |
|---|---|---|
| Active principle | Coated granules | 24.62 mg |
| | from Example 1 | equivalent to |
| | | 10 mg |
| | | of ramipril |
| Tableting agent | Mannitol | 159 mg |
| Disintegrant | Crospovidone | 22 mg |
| Permeabilizing | Precipitated | 2.2 mg |
| agent | silica | |
| Sweetener | Aspartame | 4.4 mg |
| Flavor | Orange flavoring | 2.2 mg |
| Lubricant | Magnesium | 2.2 mg |
| | stearate | |

The mixture obtained is subsequently distributed and shaped on a rotary tableting machine equipped with dies and punches 8 mm in diameter.

The hardness of the tablets obtained is approximately 30 N.

The disaggregation time in the mouth of the tablets thus obtained is less than 20 seconds.

The abrasion constitutes an important property of the tablets in accordance with the invention. It is measured according to the method described in the French pharmacopoeia (Xth edition, “V.5.1-friabilitédes comprimés”, [tablet friability], January 1993) using an apparatus having blades, and has been found to be less than 1%.

The unpleasant taste of ramipril is undetected during the residence time of the tablet in the mouth, in other words between the moment of its administration and the moment of deglutition.

The dissolution kinetics are determined according to the European Pharmacopoeia, 3rd edition, in a type II apparatus equipped with rotating blades.

For this purpose 1 tablet is introduced into this apparatus in a dissolution volume of 500 ml in 0.1 N HCl medium and stirring is carried out at a speed of 75 revolutions per minute. The concentration of ramipril dissolved in the medium is measured at regular intervals.

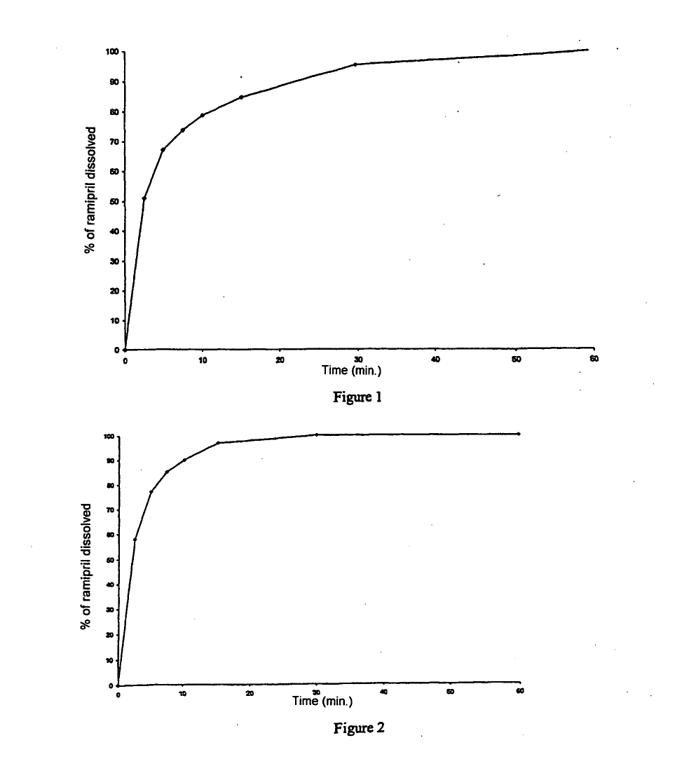

The results obtained are reported in FIG. 1 in the form of a curve C₁ which illustrates the dissolution of the active substance as a function of time.

As shown by said curve C₁, the rapid-breakdown multiparticulate tablets of ramipril allow an undelayed release of ramipril to be obtained.

### EXAMPLE 3

### Preparation of Rapid-breakdown Multiparticulate Tablets Containing 7.5 mg of Ramipril

Tablets are prepared which contain 7.5 mg of ramipril and whose composition is evident from Table III below:

TABLE III
Composition of tablets containing 7.5 mg of ramipril

| | | |
|---|---|---|
| Active principle | Coated granules | 18.53 mg |
| | from Example 1 | equivalent to |
| | | 7.5 mg of |
| | | ramipril |
| Tableting agent | Mannitol | 119 mg |
| Disintegrant | Crospovidone | 16.2 mg |
| Permeabilizing | Precipitated | 1.6 mg |
| agent | silica | |
| Sweetener | Aspartame | 3.2 mg |
| Flavor | Mint flavoring | 1.6 mg |
| Lubricant | Magnesium | 1.6 mg |
| | stearate | |

For the preparation of these tablets the procedure is as in Example 2, using the excipients and the active principle in the relative proportions which result from Table III above.

The weight of the tablets is 162 mg.

The hardness of the tablets obtained is approximately 30 N.

The disaggregation time in the mouth of the tablets thus obtained is less than 20 seconds.

The abrasion, measured as in Example 2, was found to be less than 1%.

The unpleasant taste of ramipril is undetected during the residence time of the tablet in the mouth, in other words between the moment of its administration and the moment of deglutition.

### EXAMPLE 4

### Preparation of Coated Granules of Ramipril

The granules are manufactured according to the following process.

First of all a polymer solution is prepared by dissolving Eudragit® E100 in isopropyl alcohol.

Subsequently a homogeneous powder mixture consisting of 500 g of ramipril, 750 g of mannitol, and 53 g of precipitated silica is fluidized in a fluidized-air bed.

The granulometry of the various powders is less than 100 μm.

The mixture of powders is advantageously granulated in a fluidized-air bed granulator.

Granulation is carried out by spraying the fluidized bed of the homogeneous powder mixture with a first fraction of approximately 15 to 25% of the polymer solution, for a period of approximately 30 minutes at a spraying rate of approximately 20 to 25 grams/minute under an atomizing pressure of the solution of 1.5 bar.

This gives a fluidized bed of granules whose granulometry is predominantly between 100 and 500 μm. The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2 bar.

This gives a mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 10 mg of ramipril, it will be necessary to take 31.5 mg of the homogeneous mixture of coated granules. The composition of these 31.5 mg is indicated in Table IV below:

TABLE IV

| | | |
|---|---|---|
| Active principle | Ramipril | 10 mg |
| Diluent | Mannitol | 15 mg |
| Binder/coating | Eudragit ® E 100 | 5 mg |
| agent | | |
| Antistatic agent | Precipitated | 1.5 mg |
| | silica | |
| | TOTAL | 31.5 mg |

### EXAMPLE 5

### Preparation of Rapid-breakdown Multiparticulate Tablets Containing 10 mg of Ramipril

Tablets are prepared which contain 10 mg of ramipril and whose composition is evident from Table V below:

TABLE V
Composition of a tablet containing 10 mg of ramipril

| | | |
|---|---|---|
| Active principle | Coated granules | 31.5 mg |
| | from Example 4 | equivalent to |
| | | 10 mg of ramipril |
| Tableting agent | Mannitol | 231 mg |
| Disintegrant | Crospovidone | 25 mg |
| Permeabilizing | Precipitated | 1.5 mg |
| agent | silica | |
| Sweetener | Aspartame | 5 mg |
| Flavor | Lemon flavoring | 3 mg |
| Lubricant | Magnesium | 3 mg |
| | stearate | |

For the preparation of these tablets the procedure is as in Example 2, using the excipients and the active principle in the relative proportions which result from Table V above.

The weight of the tablets is 300 mg.

The hardness of the tablets obtained is approximately 35 N.

The disaggregation time in the mouth of the tablets thus obtained is less than 25 seconds.

The abrasion, measured as in Example 2, was found to be less than 1%.

The unpleasant taste of ramipril is undetected during the residence time of the tablet in the mouth, in other words between the moment of its administration and the moment of deglutition.

The dissolution kinetics are determined according to the European Pharmacopoeia, 3rd edition, in a type II apparatus equipped with rotating blades.

For this purpose 1 tablet is introduced into this apparatus in a dissolution volume of 500 ml in 0.1 N HCl medium and stirring is carried out at a speed of 75 revolutions per minute. The concentration of ramipril dissolved in the medium is measured at regular intervals.

The results obtained are reported in FIG. 2 in the form of a curve C₂ which illustrates the dissolution of the active substance as a function of time.

As shown by said curve C₂, the rapid-breakdown multiparticulate tablets of ramipril allow an undelayed release of ramipril to be obtained.

### EXAMPLE 6

### Preparation of Rapid-breakdown Multiparticulate Tablets Containing 5 mg of Ramipril

The ramipril granules described in Example 4 are formulated to rapid-breakdown multiparticulate tablets containing 5 mg of ramipril, whose composition is given in Table VI below:

TABLE VI
Composition of a tablet containing 5 mg of ramipril

| | | |
|---|---|---|
| Active principle | Coated granules | 15.75 mg |
| | from Example 4 | equivalent to |
| | | 5 mg |
| | | of ramipril |
| Tableting agent | Mannitol | 115 mg |
| Disintegrant | Crospovidone | 12 mg |
| Permeabilizing | Precipitated | 0.7 mg |
| agent | silica | |
| Sweetener | Aspartame | 3 mg |
| Flavor | Mint flavoring | 1.5 mg |
| Lubricant | Magnesium | 1 mg |
| | stearate | |

The tablets are manufactured according to the process of Example 2, using the various excipients and the active principle in the proportions given in Table VI.

The weight of the tablets is 150 mg.

The hardness of the tablets obtained is approximately 35 N.

The disaggregation time in the mouth of the tablets thus obtained is less than 20 seconds.

The abrasion, measured as in Example 2, was found to be less than 1%.

The taste of ramipril is undetected during the residence time of the tablet in the mouth, in other words between the moment of its administration and the moment of deglutition.

### EXAMPLE 7

### Preparation of Coated Granules of Captopril

The granules are manufactured according to the following process.

First of all a polymer solution is prepared by dissolving Eudragit® E100 in isopropyl alcohol.

Subsequently a homogeneous powder mixture consisting of 750 g of captopril, 250 g of mannitol, and 53 g of precipitated silica is fluidized in a fluidized-air bed.

Granulation is carried out by spraying the fluidized bed of the homogeneous powder mixture with a first fraction of approximately 15 to 25% of the polymer solution, for a period of approximately 30 minutes at a spraying rate of approximately 20 to 25 grams/minute under an atomizing pressure of the solution of 1.5 bar.

This gives a fluidized bed of granules whose granulometry is predominantly between 100 and 500 μm. The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2 bar.

This gives a mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 50 mg of captopril, it will be necessary to take 76.9 mg of the homogeneous mixture of coated granules. The composition of these 76.9 mg is indicated in Table VII below:

TABLE VII

| | | |
|---|---|---|
| Active principle | Captopril | 50 mg |
| Diluent | Mannitol | 16.7 mg |
| Binder/coating | Eudragit ® E 100 | 6.7 mg |
| agent | | |
| Antistatic agent | Precipitated | 3.5 mg |
| | silica | |
| | TOTAL | 76.9 mg |

### EXAMPLE 8

### Preparation of Rapid-breakdown Multiparticulate Tablets Containing 50 mg of Captopril

Tablets are prepared which contain 50 mg of captopril and whose composition is evident from Table VIII below:

TABLE VIII
Composition of a tablet containing 50 mg of captopril

| | | |
|---|---|---|
| Active principle | Coated granules | 76.9 mg |
| | from Example 7 | equivalent to |
| | | 50 mg of |
| | | captopril |
| Tableting agent | Mannitol | 112 mg |
| Disintegrant | Crospovidone | 22 mg |
| Permeabilizing | Precipitated | 4 mg |
| agent | silica | |
| Sweetener | Aspartame | 5 mg |
| Flavor | Lemon flavoring | 2 mg |
| Lubricant | Magnesium | 2 mg |
| | stearate | |

For the preparation of these tablets the procedure is as in Example 2, using the excipients and the active principle in the relative proportions which result from Table VIII above.

The weight of the tablets is 224 mg.

The hardness of the tablets obtained is approximately 40 N.

The disaggregation time in the mouth of the tablets thus obtained is less than 30 seconds.

The abrasion, measured as in Example 2, was found to be less than 1%.

The unpleasant taste of captopril is undetected during the residence time of the tablet in the mouth, in other words between the moment of its administration and the moment of deglutition.

### EXAMPLE 9

### Preparation of Coated Granules of Enalapril

The granules are manufactured according to the following process.

First of all a polymer solution is prepared by dissolving ethylcellulose in isopropyl alcohol.

Subsequently a homogeneous powder mixture consisting of 500 g of enalapril, 500 g of mannitol, and 50 g of precipitated silica is fluidized in a fluidized-air bed.

Granulation is carried out by spraying the fluidized bed of the homogeneous powder mixture with a first fraction of approximately 15 to 25% of the polymer solution, for a period of approximately 30 minutes at a spraying rate of approximately 20 to 25 grams/minute under an atomizing pressure of the solution of 2 bar.

This gives a fluidized bed of granules whose granulometry is predominantly between 100 and 500 μm. The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2.5 bar.

This gives a mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 10 mg of enalapril, it will be necessary to take 23 mg of the homogeneous mixture of coated granules. The composition of these 23 mg is indicated in Table IX below:

TABLE IX

| | | |
|---|---|---|
| Active principle | Enalapril | 10 mg |
| Diluent | Mannitol | 10 mg |
| Binder/coating | Ethylcellulose | 2 mg |
| agent | | |
| Antistatic agent | Precipitated | 1 mg |
| | silica | |
| | TOTAL | 23 mg |

### EXAMPLE 10

### Preparation of Rapid-breakdown Multiparticulate Tablets Containing 10 mg of Enalapril

Tablets are prepared which contain 10 mg of enalapril and whose composition is evident from Table X below:

TABLE X
Composition of a tablet containing 10 mg of enalapril

| | | |
|---|---|---|
| Active principle | Coated granules | 23 mg equivalent |
| | from Example 9 | to 10 mg of |
| | | enalapril |
| Tableting agent | Mannitol | 120 mg |
| Disintegrant | Crospovidone | 10 mg |
| Permeabilizing | Precipitated | 1.5 mg |
| agent | silica | |
| Sweetener | Aspartame | 3 mg |
| Flavor | Mint flavoring | 2 mg |
| Lubricant | Magnesium | 1.5 mg |
| | stearate | |

For the preparation of these tablets the procedure is as in Example 2, using the excipients and the active principle in the relative proportions which result from Table X above.

The weight of the tablets is 161 mg.

The hardness of the tablets obtained is approximately 30 N.

The disaggregation time in the mouth of the tablets thus obtained is less than 25 seconds.

The abrasion, measured as in Example 2, was found to be less than 1%.

The unpleasant taste of enalapril is undetected during the residence time of the tablet in the mouth, in other words between the moment of its administration and the moment of deglutition.

### EXAMPLE 11

### Preparation of Coated Granules of Lisinopril

The granules are manufactured according to the following process.

First of all a polymer solution is prepared by dissolving Eudragit® E100 in isopropyl alcohol.

Subsequently a homogeneous powder mixture consisting of 500 g of lisinopril, 500 g of mannitol, and 50 g of precipitated silica is fluidized in a fluidized-air bed.

Granulation is carried out by spraying the fluidized bed of the homogeneous powder mixture with a first fraction of approximately 15 to 25% of the polymer solution, for a period of approximately 30 minutes at a spraying rate of approximately 20 to 25 grams/minute under an atomizing pressure of the solution of 1.5 bar.

This gives a fluidized bed of granules whose granulometry is predominantly between 100 and 500 μm. The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2 bar.

This gives a mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 20 mg of lisinopril, it will be necessary to take 49 mg of the homogeneous mixture of coated granules. The composition of these 47.2 mg is indicated in Table XI below:

TABLE XI

| | | |
|---|---|---|
| Active principle | Lisinopril | 20 mg |
| Diluent | Mannitol | 20 mg |
| Binder/coating | Eudragit ® E 100 | 5.2 mg |
| agent | | |
| Antistatic agent | Precipitated | 2 mg |
| | silica | |
| | TOTAL | 47.2 mg |

### EXAMPLE 12

### Preparation of Rapid-breakdown Multiparticulate Tablets Containing 20 mg of Lisinopril

Tablets are prepared which contain 20 mg of lisinopril and whose composition is evident from Table XII below:

TABLE XII
Composition of a tablet containing 20 mg of lisinopril

| | | |
|---|---|---|
| Active principle | Coated granules | 47.2 mg |
| | from Example 11 | equivalent to |
| | | 20 mg of |
| | | lisinopril |
| Tableting agent | Mannitol | 120 mg |
| Disintegrant | Crospovidone | 12 mg |
| Permeabilizing | Precipitated | 2 mg |
| agent | silica | |
| Sweetener | Aspartame | 3 mg |
| Flavor | Mint flavoring | 2 mg |
| Lubricant | Magnesium | 1.8 mg |
| | stearate | |

For the preparation of these tablets the procedure is as in Example 2, using the excipients and the active principle in the relative proportions which result from Table XII above.

The weight of the tablets is 188 mg.

The hardness of the tablets obtained is approximately 35 N.

The disaggregation time in the mouth of the tablets thus obtained is less than 25 seconds.

The abrasion, measured as in Example 2, was found to be less than 1%.

The unpleasant taste of lisinopril is undetected during the residence time of the tablet in the mouth, in other words between the moment of its administration and the moment of deglutition.

### EXAMPLE 13

### Preparation of Coated Granules of Trandolapril

The granules are manufactured according to the following process.

First of all a polymer solution is prepared by dissolving Eudragit® E100 in isopropyl alcohol.

Subsequently a homogeneous powder mixture consisting of 250 g of trandolapril, 750 g of mannitol, and 50 g of precipitated silica is fluidized in a fluidized-air bed.

Granulation is carried out by spraying the fluidized bed of the homogeneous powder mixture with a first fraction of approximately 15 to 25% of the polymer solution, for a period of approximately 30 minutes at a spraying rate of approximately 20 to 25 grams/minute under an atomizing pressure of the solution of 1.5 bar.

This gives a fluidized bed of granules whose granulometry is predominantly between 100 and 500 μm. The granules are then coated by spraying the fluidized bed of granules with the remaining fraction of the polymer solution, the duration of spraying being approximately 2 hours, the spraying rate being approximately 15 to 20 grams/minute, and the atomizing pressure of the solution being 2 bar.

This gives a mixture of coated granules.

The homogeneous mixture of coated granules has a statistically constant composition.

Accordingly it is possible to determine the amount of active principle present in a given mass of this homogeneous mixture of coated granules, and, conversely, it is possible, for a desired amount of active principle, to determine the corresponding amount of homogeneous mixture of coated granules.

For example, in order to have 4 mg of trandolapril, it will be necessary to take 18.4 mg of the homogeneous mixture of coated granules. The composition of these 18.4 mg is indicated in Table XIII below:

TABLE XIII

| | | |
|---|---|---|
| Active principle | Trandolapril | 4 mg |
| Diluent | Mannitol | 12 mg |
| Binder/coating | Eudragit ® E 100 | 1.6 mg |
| agent | | |
| Antistatic agent | Precipitated | 0.8 mg |
| | silica | |
| | TOTAL | 18.4 mg |

### EXAMPLE 14

### Preparation of Rapid-breakdown Multiparticulate Tablets Containing 4 mg of Trandolapril

Tablets are prepared which contain 4 mg of trandolapril and whose composition is evident from Table XIV below:

TABLE XIV
Composition of a tablet containing 4 mg of trandolapril

| | | |
|---|---|---|
| Active principle | Coated granules | 18.4 mg |
| | from Example 13 | equivalent to |
| | | 4 mg of |
| | | trandolapril |
| Tableting agent | Mannitol | 117.6 mg |
| Disintegrant | Crospovidone | 16 mg |
| Permeabilizing | Precipitated | 1.5 mg |
| agent | silica | |
| Sweetener | Aspartame | 3 mg |
| Flavor | Mint flavoring | 2 mg |
| Lubricant | Magnesium | 1.5 mg |
| | stearate | |

For the preparation of these tablets the procedure is as in Example 2, using the excipients and the active principle in the relative proportions which result from Table XIV above.

The weight of the tablets is 160 mg.

The hardness of the tablets obtained is approximately 35 N.

The disaggregation time in the mouth of the tablets thus obtained is less than 15 seconds.

The abrasion, measured as in Example 2, was found to be less than 1%.

The unpleasant taste of trandolapril is undetected during the residence time of the tablet in the mouth, in other words between the moment of its administration and the moment of deglutition.

1. Granules based on angiotensin-converting enzyme inhibitor, its isomers or its pharmaceutically acceptable salts (CEI), characterized in that they are coated and in that they comprise
CEI microcrystals,
one or more binders selected from the group comprising in particular cellulosic polymers, especially ethylcellulose, hydroxypropylcellulose, and hydroxypropylmethylcellulose, acrylic polymers, povidones, polyvinyl alcohols, and mixtures thereof,
optionally a diluent selected from the group comprising in particular cellulose derivatives, starches, lactose, polyols, especially mannitol, and an antistatic agent selected from the group comprising in particular colloidal silica, precipitated silica, and micronized or non-micronized talc.

2. Granules according to claim 1, characterized in that the CEI is selected from the group comprising in particular benazepril, captopril, cilazapril, enalapril, sosinopril, lisinopril, perindopril, quinapril, ramipril, and trandolapril, their isomers, and their pharmaceutically acceptable salts.

3. Granules according to claim 2, characterized in that the CEI is selected from the group consisting of ramipril, captopril, enalapril, lisinopril, and trandolapril, their isomers or their pharmaceutically acceptable salts.

4. Granules according to any one of claims 1 to 3, characterized in that they have a granulometry such that at least 80% of the granules have a size of between 100 and 500 μm and less than 15% of the granules have a size of less than 100 μm.

5. Granules according to any one of claims 1 to 4, characterized in that
the amount of binder is not more than 15% by weight, preferably not more than 10% by weight relative to the weight of the uncoated granules,
the amount of diluent is not more than 85% by weight, preferably not more than 50% by weight relative to the weight of the uncoated granules,
the amount of antistatic agent is not more than 10% by weight, preferably not more than 3% by weight relative to the weight of the uncoated granules.

6. Granules according to any one of claims 1 to 5, characterized in that they are coated with a coating composition comprising a coating polymer selected from the group comprising cellulosic polymers, especially ethylcellulose, hydroxypropylcellulose, and hydroxypropylmethylcellulose, acrylic polymers, and mixture thereof, and optionally an antistatic agent, plasticizers, and soluble agents, especially polyols.

7. Granules according to any one of claims 1 to 6, characterized in that the polymer constituting the binder of the uncoated granule and the coating polymer are the same.

8. Granules according to claim 7, characterized in that they comprise
from 10 to 95% of CEI microcrystals,
from 0 to 85% of a diluent, preferably mannitol,
from 0 to 10% of an antistatic agent, preferably colloidal silica,
from 5 to 10% of coating polymer or binder, preferably ethylcellulose,
the percentages being expressed by weight relative to the weight of the coated granule.

9. A process for preparing coated granules according to any one of claims 1 to 8, characterized in that it comprises in succession the steps
of dry mixing of CEI microcrystals with the diluent and, optionally, an antistatic agent,
of granulation of the mixture obtained in the preceding step by spraying of a solution or suspension of the binder,
of coating of the granules thus obtained by spraying of a suspension of the coating composition,
of drying of the coated granules thus obtained.

10. The process according to claim 9, characterized in that the various steps are performed in a fluidized-air device.

11. The process according to either of claims 9 and 10, characterized in that the spraying rate of the solution or suspension of binder in the granulation step is higher than the spraying rate of the suspension of the coating composition in the coating step and the atomizing pressure is lower in the granulation step than in the coating step.

12. A rapid-breakdown or fast disintegrating tablet of the type of those intended to undergo disaggregation in the mouth in contact with the saliva in less than 60 seconds, preferably in less than 40 seconds, forming a suspension which is easy to swallow, characterized in that it is based on coated granules according to any one of claims 1 to 8 or as prepared by the process of claims 9 to 11 and a mixture of excipients comprising at least one disintegrant, a diluent soluble agent, a lubricant, and, optionally, a swelling agent, a permeabilizing agent, sweeteners, and flavorings.

13. The tablet according to claim 12, characterized in that the disintegrant is selected from the group comprising in particular croscarmellose, crospovidone, and mixtures thereof.

14. The tablet according to either of claims 12 and 13, characterized in that the soluble agent has binding properties and consists of a polyol of less than 13 carbon atoms which is present either in the form of the directly tabletable product whose average particle diameter is from 100 to 500 μm or in the form of a powder whose average particle diameter is less than 100 μm, said polyol being preferably selected from the group consisting of mannitol, xylitol, sorbitol, and maltitol, with the proviso that the sorbitol cannot be used alone and that, where the diluent soluble agent having binding properties is alone, it is used in the form of the directly tabletable product whereas, where there are at least two diluent soluble agents having binder properties, one is present in the directly tabletable form and the other in the powder form, it being possible then for the polyol to be the same, the proportions of directly tabletable polyol and of powder polyol being from 99/1 to 20/80, preferably from 80/20 to 20/80, more preferably still from 80/20 to 50/50.

15. The tablet according to any one of claims 12 to 14, characterized in that the proportion of mixture of excipients relative to coated CEI granules is from 0.4 to 10, preferably from 1 to 10, and more preferably still from 1 to 4 parts by weight.

16. The tablet according to any one of claims 12 to 15, characterized in that the proportion of disintegrant relative to the mass of tablet is from 1 to 15% and, preferably, from 2 to 7% by weight and the proportion of soluble agent relative to the mass of the tablet is from 30 to 90% and preferably from 40 to 70% by weight.

17. The tablet according to any one of claims 12 to 16, characterized in that the permeabilizing agent is selected from the group comprising in particular silicas exhibiting a great affinity for aqueous solvents, such as precipitated silica, maltodextrins, β-cyclodextrins, and mixtures thereof.

18. The tablet according to any one of claims 12 to 17, characterized in that the lubricant is selected from the group comprising in particular magnesium stearate, sodium stearylfumarate, stearic acid, micronized polyoxyethylene glycol, and mixtures thereof.

19. The tablet according to any one of claims 12 to 18, characterized in that the sweetener is selected from the group comprising in particular aspartame, acesulfam potassium, potassium saccharinate, neohesperidine dihydrochalcone, sucralose, monoammonium glycyrrhizanate, and mixtures thereof.

20. The tablet according to any one of claims 12 to 19, characterized in that the lubricant is in powder form and is distributed for at least its major part on the surface of the tablet.
